(19) United States
(12) Patent Application Publication
(10) Pub. No.: US 2023/0363425 A1
(43) Pub. Date: Nov. 16, 2023

(54) INDUSTRIAL CARRYOVER COOKING

(71) Applicant: John Bean Technologies Corporation, Chicago, IL (US)

(72) Inventor: Richard D. Stockard, Kirkland, WA (US)

(73) Assignee: John Bean Technologies Corporation, Chicago, IL (US)

(21) Appl. No.: 18/317,829

(22) Filed: May 15, 2023

Related U.S. Application Data
(60) Provisional application No. 63/364,788, filed on May 16, 2022.

(51) Int. Cl. A23L 3/005; A23L 3/36

(52) U.S. Cl. CPC A23L 3/005; A23L 3/361

(57) ABSTRACT

A system (20) for thermal processing food products (28) includes an oven 22 operating under selected parameters. A buffer area (26) receives and holds the food products heated in the oven for a selected time period before further processing. The buffer area can be heated or cooled as needed. A temperature determining system (120) determines the temperature of the food product during the thermal processing. A control system (30) receives the operational parameters of the oven and/or the buffer area and receives the temperature determination system data. The control system uses the temperature determination system data to determine over time the temperature equilibration of the interior of the food product after being heated in the oven to model the lethality of pathogenic microorganisms in the interior of the food product. The control system can adjust one or more of the operational parameters of the oven and/or buffer area as needed to achieve a desired lethality of the pathogenic microorganisms in the interior of the food product.

### CROSS-REFERENCE TO RELATED APPLICATION

This application claims the benefit of U.S. Provisional Application No. 63/364,788, filed May 16, 2022, the entire contents of which are incorporated herein by reference.

### BACKGROUND

Typically, food products for commercial distribution, such as meat, poultry, and fish, are first cooked to a sufficient temperature (greater than 165° F. internal temperature) to kill off any pathogens that may be on the exterior or interior of the food product, such as E. coli. When the food product reaches 165° F., the bacteria is killed instantaneously. However, the large amount of heat that must be conducted through the surface to the interior of the food product to achieve the 165° F. internal temperature fully cooks the food product and results in the food product becoming relatively dry and tough when reheated for serving after commercial storage and distribution. This condition is due to juices in the food product being driven out, the denaturing of proteins, and the rendering of fats in the food product by the heat applied thereto.

Consequently, a need exists for a commercial system and method for sufficiently precooking/pasteurizing meat to eliminate pathogens, but reducing the loss of moisture from the food product and not denaturing the proteins and not rending the fats in the food product. The present disclosure seeks to address this issue.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In accordance with one embodiment of the present disclosure, a system for thermal processing of food products is provided. The system includes an oven for heating food products, the oven operating under selected operating parameters, a buffer area for receiving and holding food products removed from the oven for a selected time period before further processing of the food products, a temperature determining system comprising at least one temperature measuring device for measuring the temperature of the food product during the thermal processing of the food product and producing data related thereto, a control system including a computing device operational to receive the operational parameters of the oven and the temperature measurement data from the at least one temperature measuring device and operational to use the temperature measurement data from the at least one temperature measuring device to determine over time the temperature equilibration of the interior of the food product after leaving the oven to model the lethality of pathogenic microorganisms in the interior of the food product including after leaving the oven, and operational to adjust one or more of the operational parameters of the oven as needed to achieve a desired lethality of the pathogenic microorganisms in the interior of the food product.

In any of the embodiments described herein, wherein the temperature determining system determines the temperature of the food products after exiting the oven.

In any of the embodiments described herein, wherein the temperature determining system also determines the temperature of the food product at the buffer area.

In any of the embodiments described herein, wherein the temperature determining system comprises a first thermal imaging camera capturing food product exiting the oven.

In any of the embodiments described herein, wherein the control system processes the data from the first thermal imaging camera and using past temperature measurements of food products that had been analyzed using a machine learning model, to determine the internal temperature of the food product and the equilibration of the internal temperature of the food product over time.

In any of the embodiments described herein, wherein the temperature determining system determines the temperature of the food product in the buffer area.

In any of the embodiments described herein, wherein the thermal determining system comprises a second thermal imaging camera capturing the food products in the buffer area.

In any of the embodiments described herein, wherein the control system processes the data from the second thermal imaging camera and also uses past temperature measurements of food products that had been analyzed using a machine learning model, to determine the internal temperature of the food product and the equilibration of the internal temperature of the food product occurring prior to capture by the second thermal imaging camera.

In any of the embodiments described herein, wherein the temperature determining system comprises a first probe for determining the internal temperature food product exiting the oven.

In any of the embodiments described herein, wherein the first probe measures the internal temperature of the food product over time in the buffer area.

In any of the embodiments described herein, wherein the temperature determining system determines the temperature of the food products at one or more of the following locations: before entering the oven; within the oven; after exiting the oven; within the buffer are; at the buffer area exit.

In any of the embodiments described herein, further comprises a first conveyance to carry the food product through the oven.

In any of the embodiments described herein, further comprising a second conveyance to carry the food product through the buffer area.

In any of the embodiments described herein, wherein the control of the speed of conveyance of the food product through the buffer area is independent of the control of the speed of conveyance of the food product through the oven.

In any of the embodiments described herein, wherein the operational parameters of the oven comprise the temperature within the oven, the humidity within the oven, the composition of the processing medium in the oven, the temperature of the processing medium entering the oven, the volumetric flow rate of the processing medium flowing through the oven; the duration of time of the food product within the oven, the pressure within the oven, the dew point temperature within the oven, and the oxygen content within the oven.

In any of the embodiments described herein, wherein the buffer area comprises a holding chamber, and the second conveyance carries the food product through the holding chamber.

In any of the embodiments described herein, wherein the oven is selected from the group consisting of a convection oven, a linear convection oven, and impingement oven, a linear impingement oven, a spiral oven, a spiral convection oven, a spiral impingement oven, a microwave oven, a radio frequency oven.

In any of the embodiments described herein, wherein the food product is arranged to move in a path through the holding chamber selected from the group consisting of a linear path a serpentine path and a spiral path.

In accordance with one embodiment of the present disclosure, a time-temperature method is provided for thermal processing of a food product. The method includes subjecting the food product to thermal processing in an oven operated under selected operating parameters, transferring the food products to a buffer area, removing the food product from the buffer area for further processing, determining the temperature of the food product during the thermal processing of the food product, including the internal temperature of the food product, over time after leaving the oven, modeling a lethality of pathogenic microorganisms on and/or in the food products after removal from the oven to determine if a desired level of lethality of pathogenic microorganisms was achieved, the modeling using the determined internal temperature of the food product after leaving the oven, based on such modeling, if the desired level of lethality of pathogenic microorganisms on and/or in the food products was not achieved, adjusting one or more operational parameters of the oven.

In any of the embodiments described herein, wherein the operational parameters of the oven comprise the temperature within the oven, the humidity within the oven, the composition of the processing medium in the oven, the temperature of the processing medium entering the oven, the volumetric flow rate of the processing medium flowing through the oven, the duration of time that the food products are within the oven.

In any of the embodiments described herein, further comprising conveying the food products through the oven and through the buffer area.

In any of the embodiments described herein, wherein the speed of conveyance of the food products through the oven is independent of the speed of conveyance of the food products through the buffer area.

In any of the embodiments described herein, further comprising determining the temperature of the food products upon exiting the oven.

In any of the embodiments described herein, further comprising determining the temperature of the food products upon exiting the oven with a first thermal imaging camera.

In any of the embodiments described herein, further comprising processing the data from the first thermal imaging camera, and using past temperature measurement data of food products previously thermally processed that had been analyzed using a machine learning model, to determine the internal temperature of the food product and the equilibration of the internal temperature of the food product over time.

In any of the embodiments described herein, further comprising determining the temperature of the food products in the buffer area.

In any of the embodiments described herein, further comprising determining the temperature of the food products in the buffer area using a second thermal imaging camera.

In any of the embodiments described herein, further comprising processes the data from the second thermal imaging camera and using past temperature measurements of food products that had been analyzed using a machine learning model, to determine the internal temperature of the food product and the equilibration of the internal temperature of the food product occurring prior temperature measurement by the second thermal imaging camera.

In any of the embodiments described herein, further comprising using a first probe for measuring the internal temperature food product exiting the oven.

In any of the embodiments described herein, further comprising using the first probe to measure the internal temperature of the food product over time in the buffer area

In any of the embodiments described herein, further comprising determining the temperature of the food products at one or more of the following locations: before entering the oven; within the oven; after exiting the oven; within the holding chamber; after exiting the holding chamber.

In any of the embodiments described herein, further comprising subjecting the food products to thermal process in an oven, wherein the oven is selected from the group consisting of a convection oven, a linear convection oven, and impingement oven, a linear impingement oven, a spiral oven, a spiral convection oven, a spiral impingement oven, a microwave oven, a radio wave frequency oven.

In any of the embodiments described herein, further comprising conveying the food products along in a path through the buffer area selected from the group consisting of a linear path, a serpentine path, and a spiral path.

In any of the embodiments described herein, wherein the path through the buffer area extends through a holding chamber.

In accordance with one embodiment of the present disclosure, a system is provided for thermal processing of food products. The system includes an oven for heating food products, the oven operating under selected operating parameters, a buffer area operating under selected operational parameters for receiving and holding food products heated in the oven for a selected time period before further processing of the food products, a temperature determining system comprising at least one temperature measuring device for measuring the temperature of the food product during the thermal processing of the food product and producing data related thereto, a control system comprising a computing device, the control system being operational to receive the operational parameters of at least one of the oven and the buffer area, and to receive the temperature measurement data, and operational to use the temperature measurement data to determine over time the temperature equilibration of the interior of the food product after leaving the oven to model the lethality of pathogenic microorganisms in the interior of the food product, and operational to adjust one or more of the operational parameters of at least one of the oven and buffer area as needed to achieve a desired lethality of the pathogenic microorganisms in the interior of the food product.

In any of the embodiments described herein, wherein the temperature determining system determines the temperature of the food products at one or more of the following locations: before entering the oven; within the oven; after exiting the oven; within the buffer area; at the buffer area exit; exterior to the buffer area.

In any of the embodiments described herein, wherein the temperature determining system comprises at least one thermal imaging camera.

In any of the embodiments described herein, wherein the control system processes the data from the at least one thermal imaging camera and using past temperature measurements of food products that had been analyzed using a machine learning model to determine the internal temperature of the food product and the equilibration of the internal temperature of the food product over time.

In any of the embodiments described herein, wherein the thermal determining system comprises a thermal imaging camera capturing the food products in the buffer area.

In any of the embodiments described herein, wherein the control system processes the data from the thermal imaging camera and also uses past temperature measurements of food products that had been analyzed using a machine learning model to determine the internal temperature of the food product and the equilibration of the internal temperature of the food product occurring prior to capture by the thermal imaging camera.

In any of the embodiments described herein, wherein the temperature determining system comprises at least one probe for determining the internal temperature of a food product.

In any of the embodiments described herein, further comprising a conveyance to carry the food product through the oven and through the buffer area.

In any of the embodiments described herein, wherein the control of the speed of conveyance of the food product through the buffer area is independent of the control of the speed of conveyance of the food product through the oven.

In any of the embodiments described herein, wherein the operational parameters of the oven comprise the temperature within the oven, the humidity within the oven, the composition of the processing medium in the oven, the temperature of the processing medium entering the oven, the volumetric flow rate of the processing medium flowing through the oven; the duration of time of the food product within the oven, the pressure within the oven, the dew point temperature within the oven, and the oxygen content within the oven.

In any of the embodiments described herein, wherein the buffer area comprises a holding chamber, and the conveyance carries the food product through the holding chamber.

In any of the embodiments described herein, further comprising a source of thermal processing fluid for supplying the holding chamber.

In any of the embodiments described herein, wherein the temperature of the thermal processing fluid for the holding chamber is above or below 32 F.

In any of the embodiments described herein, wherein the source of thermal processing fluid comprises heating system and cooling system for supplying heated and/or cooled thermal processing fluid to the holding chamber.

In any of the embodiments described herein, wherein the holding chamber divided into a heating zone and a cooling zone, wherein the heating system and cooling system supplies heated thermal processing fluid to the heating zone and/or supplies cooled thermal processing fluid to the cooling zone

In any of the embodiments described herein, further comprising a freezer located downstream of the buffer area, the freezer operating under selected operational parameters.

In any of the embodiments described herein, wherein the control system controlling the operational parameters of the freezer.

In any of the embodiments described herein, wherein heat generated during the operation of the freezer is supplied to the buffer area.

In accordance with one embodiment of the present disclosure, a time-temperature method is provided for thermal processing of a food product. The method includes subjecting the food product to thermal processing in an oven operated under selected operating parameters, transferring the food products to a buffer area operated under one or more selected operating parameters, removing the food product from the buffer area for further processing, determining the temperature of the food product during the thermal processing of the food product, including the internal temperature of the food product, over time after being heated in the oven, modeling a lethality of pathogenic microorganisms on and/or in the food products after being heated in the oven to determine if a desired level of lethality of pathogenic microorganisms was achieved, the modeling using the determined internal temperature of the food product after leaving the oven, based on such modeling, if the desired level of lethality of pathogenic microorganisms on and/or in the food products was not achieved, adjusting one or more operating parameters of the oven and/or the buffer area.

In any of the embodiments described herein, further comprising suppling thermal processing medium to the buffer area to assist in achieving a desired level of lethality of the pathogenic microorganisms

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a schematic view of an embodiment of the present disclosure;

FIG. 2 is a schematic view of another embodiment of the present disclosure;

FIG. 3 is a schematic view of another embodiment of the present disclosure;

FIG. 4 are time/temperature graphs of chicken fillets removed from a cooking oven at different internal temperatures of the fillets; and

FIG. 5 is a schematic view of another embodiment of the present disclosure.

### DETAILED DESCRIPTION

The present application and claims relate to the continuous thermal processing of food products, by, for example, cooking, while also killing or eliminating pathogenic microorganisms that may be present on and/or in food products. The application also describes the killing of “bacteria” in and/or on food products. Such references to bacteria and pathogenic microorganisms relate to food pathogens, including, among others, the following: E. coli, Salmonella spp., Clostridium botulinum, Staphylococcus aureus, Campylobacter jejuni, Yersinia enterocolitica and Yersinia pseudotuberculosis, Listeria monocytogenes, Vibrio cholerae O1, Vibrio cholerae non-O1, Vibrio parahaemolyticus and other vibrios, Vibrio vulnificus, Clostridium perfringens, Bacillus cereus, Aeromonas hydrophila and other spp., Plesiomonas shigelloides, Shigella spp., miscellaneous enterics, and Streptococcus.

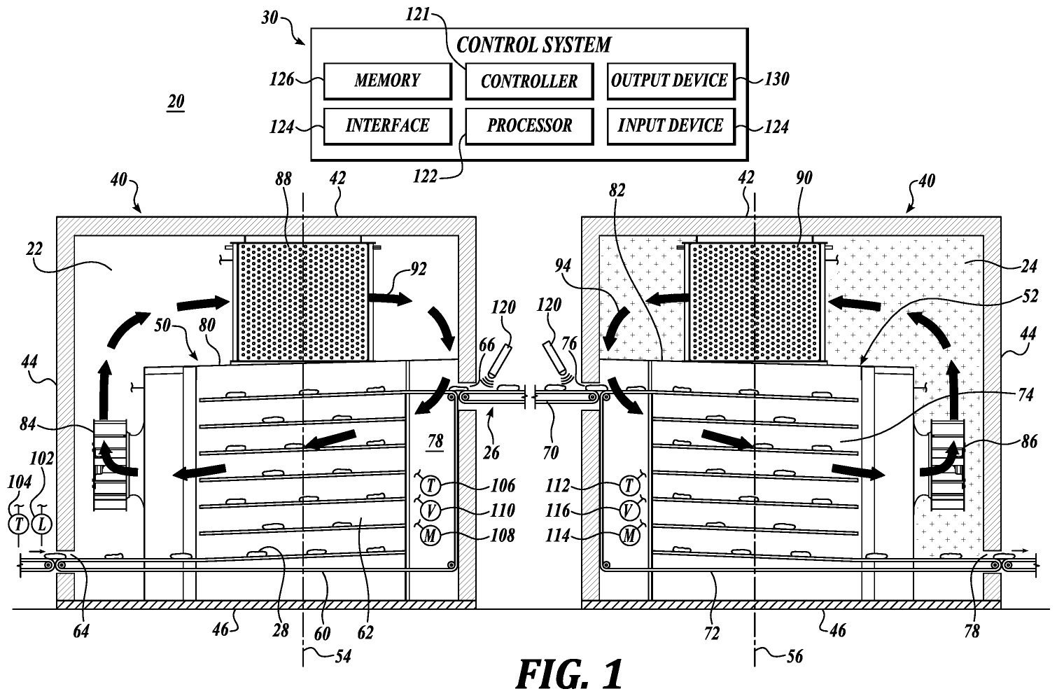

FIG. 1 schematically illustrates an embodiment of a continuous food thermal processing system 20 of the present disclosure as including a first thermal processing apparatus in the form of an oven 22 operating under selectable operating parameters and a second thermal processing apparatus in the form of a freezer 24 also operating under selectable operating parameters. A conveyor 26 transports food items 28 from the oven 22 to the freezer 24 and also serves a buffer zone or area to allow the food items from the oven to continue to be thermally processed, including allowing heat energy to continue to move toward the interior of the food item. It this manner, the temperature of the food item throughout the interior or thickness of the food item equilibrates. The conveyor 26 can be enclosed in a tunnel structure.

The temperature of the food item 28 leaving the oven 22 is measured so as to determine the temperature of the interior of the food item as well as the change in the temperature of the interior of the food item over time, i.e., during the temperature equilibration that occurs after leaving the oven. As discussed below, the temperature measurement can occur by various means, including, for example, with thermal imaging cameras or probes. Features or data from the thermal imaging camera can be provided to a temperature prediction machine learning model in order to predict the internal temperature of the food product as well as to predict the change in the internal temperature of the food product occurring during the equilibration period. This information can be used to model the level of killing or elimination of the pathogenic microorganisms within the food product achieved. If the lethality achieved is insufficient, the operating parameters of the oven and/or the buffer can be adjusted. Likewise, if the lethality achieved is more than sufficient, the operating parameters of the oven and/or the buffer can be adjusted accordingly.

The system 20 seeks to use the heat energy within the food product to continue to kill pathogens within the food products after the food product has left an oven or other thermal processing device or system. Heretofore, the lethality level of pathogenic microorganisms in a food product was based on the time that the food product was within the oven, and no consideration was given to the continued killing of pathogenic microorganisms using time/temperature lethality calculations once the food product left the oven.

The present disclosure seeks to take into consideration the elevated temperature within the interior of the food product after leaving the oven, and thus the continued killing of pathogenic microorganisms with the food product. This leads to several advantages, including a potential increased product throughput since the food product is removed from the oven at an early stage. Also, overcooking of food product is reduced, so more moisture is retained in the food product, thereby improving the quality (including moistness and tenderness) of the food product as well as its net weight. In addition, less energy is expended in cooking the food product as well as in freezing the food product after cooking, since the food product is not heated to as high a temperature within the oven as conventionally would be the case.

A control system 30 is employed to control the operation of the thermal processing system 20 to help ensure that the system 20 operates to kill a desired percentage of any pathogenic microorganisms which may be present on the surface and/or in the interior of food product 28. The control system 30 receives input from various measurement devices or instruments that measure/monitor, among other parameters, the temperature of the food products exiting the oven 22, whether determined via thermal imaging camera, probes inserted into the food product, or other means. Other parameters that may be measured and monitored include the temperature within the oven, the moisture content of the thermal processing medium within the oven, the flow rate of the thermal processing medium through the oven, the length of time the food products are in the oven, the level of loading of food products on the conveyor system 26, the temperature of the food products entering the oven, the length of time the food products are on/in the buffer, the temperature of the food items at the end of the buffer etc., as discussed more fully below. Depending on the lethality of the microorganisms achieved, one or more of these parameters can be adjusted, as discussed below

Describing the thermal processing system in more detail, thermal processing apparatus 22 (oven) and 24 (freezer) are each illustrated in FIG. 1 as including a generally rectangularly shaped housing 40 each having a top section or ceiling 42, side sections or walls and end walls 44, as well as a floor 46. The housing 40 is sized to contain first and second spiral or helical conveyor units 50 and 52.

A continuous powered conveyor belt 60 for carrying work products 28 through the apparatus 22 is arranged in tiers forming an ascending spiral stack 62 in conveyor unit 50. The conveyor belt 60 enters the spiral conveyor unit 50 at the bottom thereof at an inlet 64 in wall 44 and then travels in a spiral path until reaching the top of the spiral stack 62, and then extends tangentially from the top of stack 62 and out through an exit opening 66 to deliver the food products to a buffer conveyor 70 extending between the oven 22 and the freezer 24.

A continuous powered conveyor belt 72 for carrying work products 28 through the apparatus 24 is arranged in tiers forming a descending spiral stack 74 in conveyor unit 52. The conveyor belt 72 extends outwardly through an inlet opening 76 in wall 44 to receive the food product from the buffer conveyor 70. The conveyor belt travels in the descending spiral path of the stack 74 along the spiral conveyor unit 52 to eventually exit the freezer unit 24 from the bottom tier of the stack 74 through outlet 78 formed in wall 44. The inlets 64 and 76 and the outlets 66 and 78 can be substantially sealed from the ambient by air knives or other known means.

The centers of the conveyor stacks 62 and 74 extend around a central drive system that rotates the conveyor units 50 and 52 about a central axis 54 and 56 respectively. The drive system includes a cylindrical drive drum that frictionally and rotationally drives conveyor belts 60 and 72 over supports that are fixed in place exterior to the drum, thereby to rotate the belt about axis 54 and 56. The belts 60 and 72 tighten around the drive drum, creating enough friction therebetween to drive the belts 60 and 72 forward to slide over the supports in a standard manner.

A top panel structure 80 overlies the conveyor stack 62 and a corresponding top panel structure 82 overlies the conveyor stack 74. Circulation fans 84 and 86 are positioned at outward sides of the conveyor units 50 and 52 to draw processing medium, for example, air, across the interior of the conveyor stacks 62 and 74 (and around the drive drums) so as to thermally treat the work products being carried on the conveyor belts 60 and 72, and then direct such processing medium upwardly along the walls 44 of the housings 40 toward the ceiling 42 of the housings. Thereafter, the processing medium is directed through heat exchangers 88 and 90 positioned on or above the top panel structures 80 and 82. The processing medium extends transversely across the top of each of the stacks 62 and 74. The heat exchangers 88 and 90 may be mounted on or just above the top panel structures 80 and 82 by an appropriate mounting structure.

Thermal processing air or other thermal processing medium being circulated by the fan 84 and 86, when passing through the heat exchanger 88, is heated as desired, and the thermal processing air being circulated by fan 86, when passing through the heat exchanger 90, is cooled as desired. The heated or cooled processing medium flows horizontally over the top panel structures 80 and 82 until reaching the respective opposite side wall 44, wherein the processing medium is deflected downwardly to flow along the exposed adjacent portion of the conveyor stacks 62 and 74 and enter into the stacks in a lateral direction, as depicted by arrows 92 and 94, thereby heating/cooling the work product 28 primarily by convection heat transfer.

It is to be understood that the oven 22 and freezer 24 do not need to be constructed in a corresponding manner as described above. Further, either the oven and/or the freezer can be of different construction, for example, wherein the conveyor belt moves in a linear, serpentine, or other path.

As shown in FIG. 1, the buffer conveyor 70 transfers food product 28 from the oven 22 to the freezer 24. The buffer conveyor is constructed separately and operates independently from conveyors 50 and 52. The buffer conveyor has its own drive and control system. In this manner, the length of time that the food product is retained on the buffer conveyor can be controlled. As a result, the length of time that the temperature of the food product is allowed to equilibrate after leaving the oven 22 can be controlled.

The control system 30 is employed to control the operation of the thermal processing system 20 to help ensure that the work products (food products 28) are properly thermally processed or treated. For example, that the food products 28 are sufficiently cooked or otherwise processed so that pathogenic microorganisms which may be present on the surface and/or in the interior of food product 28 are killed to a sufficient level. It may also be desirable to achieve desired qualities consisting of sensory attributes such as color, flavor, texture, mouthfeel, etc.

Consequently, the control system 30 receives input signals from various measurement devices or instruments of a monitoring system that monitors, among other process parameters, the temperature, air/vapor mixture, velocity, and moisture content within the oven 22 and freezer 24. Other parameters that may be measured and monitored include, for example, the temperature of the food product 28 at one or more locations along the thermal processing system, the speed of the various conveyor belts 60, 70, and 72, and the level of loading of food products on the conveyor belts, as discussed more fully below.

As noted in the prior paragraph, the measuring system of the system 20 measures the operational parameters of system, including the loading frequency or density of the food product 28 loaded onto the conveyor belt 60 from a delivery conveyor, not shown. Such load monitor or sensor is schematically symbolized by the load monitor/sensor 102 shown in FIG. 1. The load monitor/sensor 102 can take various forms, including a scale to weigh the food product being transferred to conveyor belt 60. The information from the load monitor 102 is transmitted to the control system 30.

Alternatively, the load monitor can be in the form of an optical scanner capable of scanning the food product and determining the volume of the food product, then calculating the weight of the food product by using the known density of the food product. Such scanning systems are well known in the art. For example, see U.S. Pat. No. 7,452,466. The disclosure of this patent is incorporated herein by reference.

Further, if a thermal imaging camera 104 is used to determine the temperature of the food product 28 entering the oven 22, the thermal imaging camera can also be trained to measure the size of the food products 28, at least the two-dimensional area of the food products.

Regardless of the specific technology used to determine the loading level of the food product, the technology needs to be capable of determining the loading level when the food items are spaced apart as well as when load in mass, for example, as a continuous layer. This might be the case with vegetables or smaller food products, such as chicken wings.

The measuring system can also measure the temperature and moisture level within the interior of the oven 22 and the freezer 24, as well as the velocity of the air/vapor mixture flowing through the oven and freezer. These operational parameters can be measured by a temperature sensor 106, a moisture sensor 108, and a fluid velocity sensor 110 within the oven 22. Correspondingly, a temperature sensor 112, a moisture sensor 114, and a fluid velocity sensor 116 can be utilized within the freezer 24. These sensors are in communication with the control system 30, which can be by hard wiring or wireless transmission. Of course, alternative and/or additional sensors can be employed, including sensors for example, pressure sensors, dew point sensors, and oxygen level sensor. These additional sensors also provide data regarding the operating conditions of the oven and freezer.

The temperature sensor 106 is preferably configured to sense the dry bulb and wet bulb temperatures within the oven and freezer. The reason for also measuring the wet bulb temperature is that as the food product is carried through the oven 22, its surface temperature gradually increases. Eventually, this surface temperature will reach the dew point temperature of the moist, hot air in the processing zone. At that point, the moisture in the heating medium within the processing zone will not condense on the surface of the food products. Instead, the moisture on the surface of the food product will begin to evaporate, which tends to cool the food product somewhat. The temperature at which this transition occurs will be the wet bulb temperature. Nonetheless, the energy delivered to the surfaces of the food product must still be sufficient to thermally process the food product to the desired target temperature while achieving sensory attributes sought and also kill the desired level of pathogens on and/or in the food product 28. As an alternative, the monitoring system can measure the dry bulb temperature and humidity level in the processing zones. From this information it is possible to determine the wet bulb temperature, relative humidity, and dew point within the processing zones.

The measuring system can also be configured to determine the temperature of the food products leaving the oven and being transported to the freezer by the buffer conveyor. It is desired to know the interior temperature of the food product at this point in the thermal processing of the food product. In accordance with the present disclosure, one manner of achieving this end is to use a thermal imaging camera such as camera 120 schematically shown in FIG. 1. The camera 120 is able to view the distribution of the temperature of the exterior of the food product about the surface area of the food product. In this regard, food items of different thicknesses will exhibit different temperature distributions or patterns on their exterior due to heat being transferred into the interior of the food item. For example, at thinner areas of the food item, the temperature will be higher than at thicker areas of the food product.

A thermal image of a food product is captured by the thermal imaging camera. The thermal image is provided to an image segmentation machine learning model that segments the thermal image to identify pixels that depict the food product. The thermal image may be captured after the food product is transferred from the oven conveyor belt 60 (which is heated by the oven) to the buffer conveyor, so that the greater temperature differential between the food product and the conveyor can aid in segmentation.

Features extracted from the segmented thermal image (including, but not limited to, the measured surface temperature of the food product) are provided to a temperature prediction machine learning model in order to predict the internal temperature of the food product. Along with the measured surface temperature, additional features, may include one or more of: the area of the food product taken from above: the total surface area of the food product; the thickness of the food product; the thickness distribution of the food product; the volume of the food product; the volume distribution of the food product; a SKU of the food product (e.g., a type or cut, such as chicken vs. beef, or breast meat vs. thigh meat); a type of target product, such as chicken patties vs. chicken nuggets vs. popcorn chicken); whether the food product is a bone-in food product; if a bone-in food product, the size and location of the bone; cooking time or duration; oven temperature; humidity within the oven, as well as other physical features or measurements of the food product may be provided to the temperature prediction machine learning model. Training data for the temperature prediction machine learning model may be obtained by using temperature probes to generate ground truth internal temperature information.

Moreover, the temperature probes may monitor the temperature change over time within the interior of the food product to ascertain how the temperature of the food product equilibrates over time. This information can be associated with the thermal image captured by the thermal imaging camera as well as with other information about the food product noted above. This information can be used to analyze on a time-temperature basis the pathogenic organism kill level achieved during the thermal processing of the food product. This information may also be used to determine if the food product has been sufficiently heated to eliminate or reduce red color or “bone blood” or myowater. One source of bone blood is from myoglobin leaking into poultry meat due to the porosity of the bones or a broken bone. According to one reference, the red color is eliminated if the poultry is heated to above 170 Degrees F.

The temperature prediction machine learning model may be a regression-based model and may include features that represent the thermodynamics of the cooking/cooling/energy distribution process. Output of the temperature prediction machine learning model may be presented to an operator on a dashboard as a trend line in order to identify problems and/or other issues that may arise within the processing line, and/or to provide information that may allow the operator to adjust one or more aspects or parameters of the processing line (including, but not limited to, oven temperature, oven humidity level, oven conveyor speed, buffer conveyor speed, etc.).

The output of the temperature prediction machine learning model may alternatively, or in addition, cause the control system to automatically adjust one or more operating parameters of the thermal processing system 20. Further, the control system may send a warning or alert to the operator or even cause the thermal processing system to cease operation.

As schematically depicted in FIG. 1, the control system 30 includes a controller 121 for use in controlling the operation of thermal processing system 20. The control system also includes a processor/computing device 122 linked to the controller. An appropriate interface 124 is provided for connecting the various gauges, measuring devices, monitors, and components of the system 20 to the controller 121. A memory unit 126 is provided for storing information, programs, for example, the machine learning model, pertaining to the system 20. Other information in memory can include the process parameters for the type of food product being processed and desired end parameters for the fully processed food product. Further, past performance metrics of the system 20 may be stored in memory as well as maintenance records and schedule for the system 20.

A keyboard, touch screen, or other input device or interface 124 is provided to enable the operator to communicate with the processor and controller. Also, a display or other output device 130 is provided to convey information from the processor or control system to the operator, including the functioning of and operational metrics of the system 20. An example of a processor-operated control system for controlling a cooking apparatus is disclosed by U.S. Pat. No. 6,410,066, which is incorporated herein by reference.

The measuring system measures, and the control system 30 controls, the components of the system 20 so as to operate within set point parameters, including, for example, the speed of the conveyor belts 60, 70, 72; the speed of the circulation fans 84 and 86; the operation, including the thermal output, of the heat exchangers 88 and 90. These components are in communication with the control system 30. This can be accomplished by wired connection or wirelessly.

The control system 30, more specifically the processor/computing device 122 together with the controller 121, controls the various components and subsystems of system 20, including the level of the loading of the food product 28 onto the conveyor belt 60, by, for example, controlling the operation of a loading conveyor that delivers the food product to the conveyor belt. The control system 30 also controls the speed of the conveyor belts 60 and 72 by controlling the conveyor drive system. The speed of the conveyor belts 60 and 72 affects the dwell time of the work products in the oven 22 and freezer 24.

In addition, the control system controls the temperature and moisture level within the oven by controlling temperature, quality, and volume flow rate of the thermal processing medium circulated through the oven and the cooking duration. Further, the control system controls the length of time the food products have spent on the buffer conveyor, and thus the amount of time provided for the temperature of the food product to equilibrate after leaving the oven. This parameter is used by the processor 122 to determine if a desired kill level of the pathogens in, as well as on, the food product has been reached, and if not, what action should be taken as a result.

In this regard, the lethality level can be increased or decreased by adjusting the belt speed and thus the retention time of the food product in the buffer area or chamber. This alone will have minimal effect on heating or cooling the products in the buffer area, and have no effect on all other products in the oven. No changes to either internal or external heat transfer in the product is required and therefor the corrective action is simple and predictable. This provides the ability to alter the lethality by controlling and adjusting the dwell time of the food product in the buffer area/chamber, without altering the cooking of the product.

The control system 30 may be located at the thermal processing system or at a remote location. The control system may be connected to a network, whereby the thermal processing system may be monitored and/or controlled by personnel located remotely, for example, by using a remote work station, or a smart tablet, smart phone, or other smart device.

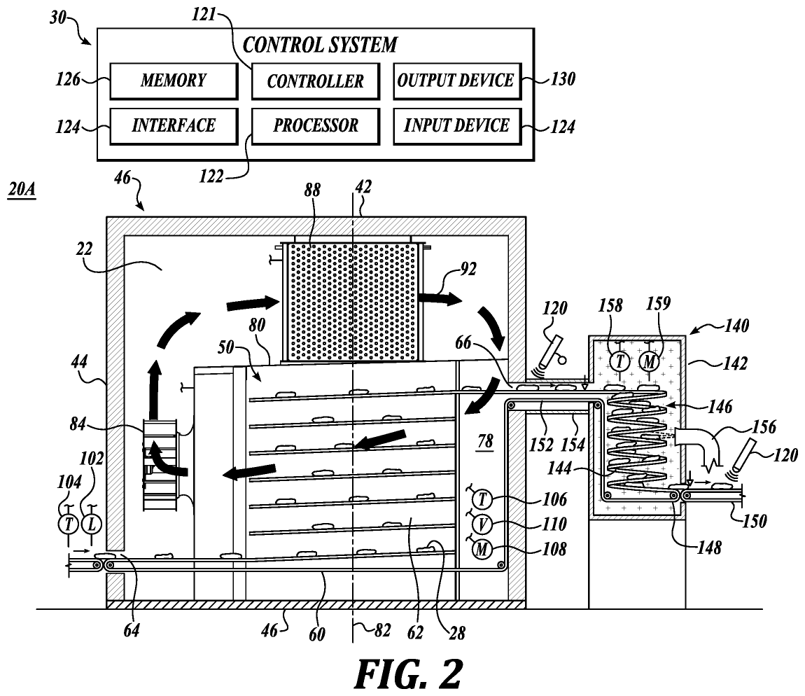

FIG. 2 schematically shows another embodiment of the present disclosure wherein in the thermal processing system 20A, the freezer 24 shown in FIG. 1 has been replaced by a buffer chamber 140 for holding the food products 28 while the temperature of the food products equivalate after leaving the oven 22. Of course, a freezer, such as freezer 24, or other processing apparatus/station may be located downstream of the buffer chamber 140.

The components of the thermal processing system 20A that are the same or similar to those of the thermal processing system 20 are identified by the same part numbers, and their description will not be repeated here.

The buffer chamber 140 is in the form of a housing 142 for containing an endless buffer conveyor 144. Within the housing 142, the buffer conveyor travels along a spiral path forming a descending stack 146 to form or define a bottom end location 148 that is end-to-end with a take away conveyor 150. The take away conveyor 150 transports the food products to a downstream processing location.

The buffer conveyor also includes upstream section 152 that extends across to the oven 22 to carry the heated food products 28 from the oven to the buffer chamber 140. Optionally, a tunnel or enclosure 154 can extend along the conveyor upstream section 152 for protection of the food products 28 being transported.

As in the thermal processing system 20, a thermal camera 120 is employed in thermal processing system 20A to determine the internal temperature of the food product leaving the oven, for example, just downstream of the outlet 66 of the oven 22. The thermal camera 104 in FIG. 2 performs the same function in FIG. 1, as described above.

It can be appreciated that the buffer chamber 140 can provide a significant buffer capacity within a relatively small volume compared to a linear length of conveyor. As such, the use of the buffer chamber enables a relatively large amount of volume of food product throughput while still providing sufficient time for the temperature of the food product to equilibrate after leaving the oven 22.

Optionally, thermal processing heating medium can be supplied to the buffer chamber 140 by a supply duct 156. This option can be useful from the data received from the thermal imaging camera 120 if it is determined that the desired lethality of the pathogens in the food product 28 has not been achieved. In this case, the housing 142 or a portion of the housing 142 could be sufficiently heated to achieve the desired kill level of the pathogens in the food product 28.

As in the thermal processing system 20, a control 30 controls the operation of the thermal processing system 20A in the manner described above, including the speed of the buffer conveyor 144. Additionally, the control system controls whether or not thermal processing heating or cooling medium is supplied to the buffer chamber and the quality of the heating medium, including, for example, the temperature of the heating medium, the moisture level of the hearing medium, the flow rate of the heating medium.

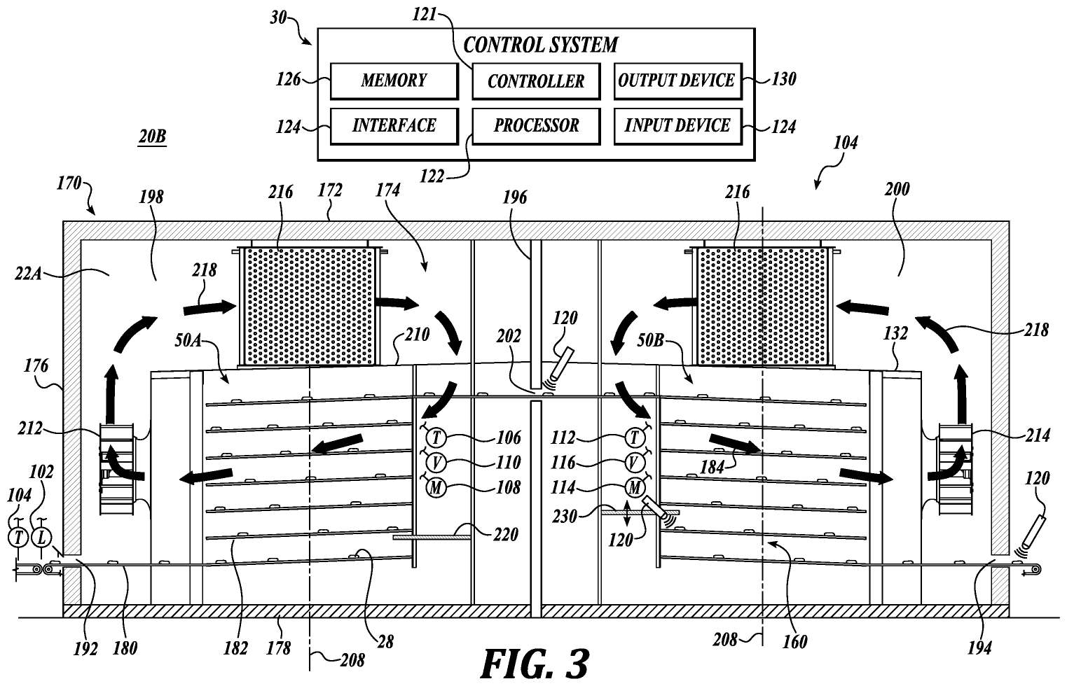

FIG. 3 schematically shows another embodiment of the present disclosure wherein, in the thermal processing system 20B, an oven 22A is composed of a first ascending spiral conveyor unit 50A and a second descending spiral conveyor unit 50B. A buffer 160 section is incorporated into the lower tiers of the descending spiral conveyor unit 50B.

The thermal processing system 20B includes a generally rectangularly shaped housing 170 having a top section or ceiling 172, longitudinal side sections or walls 174, and transverse end sections or walls 176, as well as a floor 178. The housing 170 is sized to contain first and second spiral or helical conveyor units 50A and 50B.

An endless, continuous powered conveyor belt 180 for carrying food products through the system 20B is arranged in tiers forming an ascending spiral stack 182 in conveyor unit 50A and arranged in tiers forming a descending spiral stack 184 in conveyor unit 50B. The conveyor belt 180 enters the spiral conveyor unit 50A at the bottom thereof by passing through an inlet opening 192 in wall 176, and then travels in a spiral path until reaching the top of the spiral stack 182, and then extends tangentially from the top of stack 182 to the top stack 184 to descend along the spiral conveyor unit 50B, to eventually exit the unit 50B from the bottom tier of the stack 184 through outlet opening 194 formed in wall 176. The inlet 192 and outlet 194 openings can be substantially sealed from the ambient by air knives or other means.

A center or mid wall 196 divides the two spiral conveyor units 50A and 50B into separate compartments 198 and 200, wherein different process media conditions can be employed. For example, the temperature of the air, air/vapor mixture, steam, or other processing medium, the moisture content in the air, the medium velocity, etc., may be different in the two compartments created by the mid or center wall 196. A close fitting opening 202 is provided in center wall 196 to allow passage of the conveyor belt 180 and the food products being carried thereon. If needed, an air knife or similar/other sealing system can be used to provide a seal between the two compartments 198 and 200.

The center of the conveyor stacks 182 and 184 extend around a central drive system that rotates the conveyor units 50A and 50B about a central axis 208. The drive system includes a cylindrical drive drum that frictionally and rotationally drives conveyor belt 180 over supports that are fixed in place exterior to the drum, thereby to rotate the belt about axis 208. The belt 180 tightens around the drive drum, creating enough friction therebetween to drive the belt forward to slide over the supports.

A top panel structure 210 overlies the conveyor stacks 182 and 184. Circulation fans 212 and 214 are positioned at outward sides of the conveyor units 50A and 50B to draw processing medium, for example, air, across the interior of the conveyor stacks 182 and 184 (and around drum) so as to thermally treat the food products being carried on the conveyor belt 180 and then direct such processing medium upwardly along the end walls 176 of the housing 170 toward the ceiling 172 of the housing. Thereafter, the processing medium is directed through a heat exchanger 216 positioned on or above the top panel structure 210. The processing medium extends transversely across the top of each of the stacks 182 and 184. The heat exchanger 216 may be mounted on or just above the top structure 210 by an appropriate mounting structure.

The thermal processing air or other thermal processing medium being circulated by the fans 212 and 214, when passing through the heat exchanger 216, is heated to a level as desired. The heated medium flows horizontally over the top structure 210 until reaching the center wall 196, wherein the processing medium is deflected downwardly to flow along the exposed adjacent portion of the conveyor stacks 180 and 182 and enter into the stacks in a lateral direction, as depicted by arrows 218, thereby heating the food product primarily by convection heat transfer.

The spatial arrangement of the fans 212 and 214, the heat exchanger 216, and optimally positioned center wall 196 in relation to the respective stacks enables flow distribution to be uniform to each tier within the individual stacks to deliver an air flow mixture to approach the surfaces of the food items on the conveyor diagonally. This serves as a unique feature with respect to conventional twin-drum ovens manufactured by others.

A first mezzanine 220 may be located in the lower portion of chamber or compartment 198 to effectively divide the conveyor stack 182 into a lower first processing zone below the mezzanine and an upper second processing zone above the mezzanine. The mezzanine extends outwardly from the center wall 196 toward the conveyor stack 182. The central portion of the mezzanine is scalloped or otherwise relieved to provide clearance for the conveyor stack 182. The remainder of the mezzanine 220 (located outwardly of the conveyor stack 182) extends towards the center of the conveyor stack to approximately the location of the drive drum.

The first mezzanine 220 is shown as substantially planar in construction. Appropriate braces or reinforcements can be used to provide the needed structural integrity of the mezzanine.

Optionally, the mezzanine 220 is adjustable in elevation by an appropriate actuating system. Preferably, the actuation system is powered so the control system is able to alter the height of the mezzanine as desired. The first mezzanine 220 is shown as located at an elevation between the first and second lowest tiers of the conveyor stack 182. However, the mezzanine 220 can be positioned at a higher elevation, for example, at the level of the second lowest tier or at a level above the second lowest tier of the conveyor stack 182.

A second mezzanine 230 is located in the lower portion of chamber or compartment 200 to effectively divide the conveyor stack 182 into an upper processing zone and a lower buffer section or zone 160. The mezzanine 230 extents outwardly from the center wall 196 toward the conveyor stack 182. The central portion of the mezzanine 230 is scalloped or shaped to provide clearance for the conveyor stack 182. The remainder of the mezzanine extends towards the center of the conveyor stack to approximately the location of the drive drum. In this manner, the mezzanine 230 prevents the processing medium, as depicted by arrows 218, from entering the ties of the stack 184 located below the mezzanine.

The second mezzanine 230 is also shown as substantially planar in construction. Appropriate braces or reinforcements can be used to provide the needed structural integrity of the mezzanine.

The mezzanine may be adjustable in elevation, via an appropriate actuation system. Preferably, the actuation system is powered so the control system 30 is able to alter the height of the second mezzanine as desired. The second mezzanine 230 is shown as located at an elevation between the second and third lowest tiers of the conveyor stack 182. However, the mezzanine 230 can be positioned at a higher elevation, for example, at the level of the third lowest tier or above the third lowest tier. In this manner, the size of the buffer zone 160 can be adjusted so the length of time provided for the food items to equilibrate in temperature can be selected to achieve a desired kill level of the pathogens within the food products. It will be appreciated that the buffer zone 160 is incorporated into the structure of the oven 22A. In this regard, the buffer zone 160 can be built into an existing two-tier oven such as oven 22A or other ovens.

As in the thermal processing systems 20 and 20A, one or more thermal imaging cameras 120 can be employed in thermal processing system 20B to serve the same function as in processing systems 20 and 20A, including to determine the external and internal temperature of the food products being processed. A thermal camera can be located in the buffer zone 160, preferably just downstream of the second mezzanine 230, so as to be positioned immediately after exiting the thermal processing zone of compartment 200. Thermal imaging cameras can also be located in compartment 200 just inside the center wall opening 202 and/or just outside the compartment 200 at exit opening 194.

As in the thermal processing systems 20 and 20A, a control system 30 controls the operation of the thermal processing system 20B in the manner described above, including the speed of the conveyor 180. Additionally, the control system controls the level of the second mezzanine, and thus the time the food products are located in the buffer zone 160.

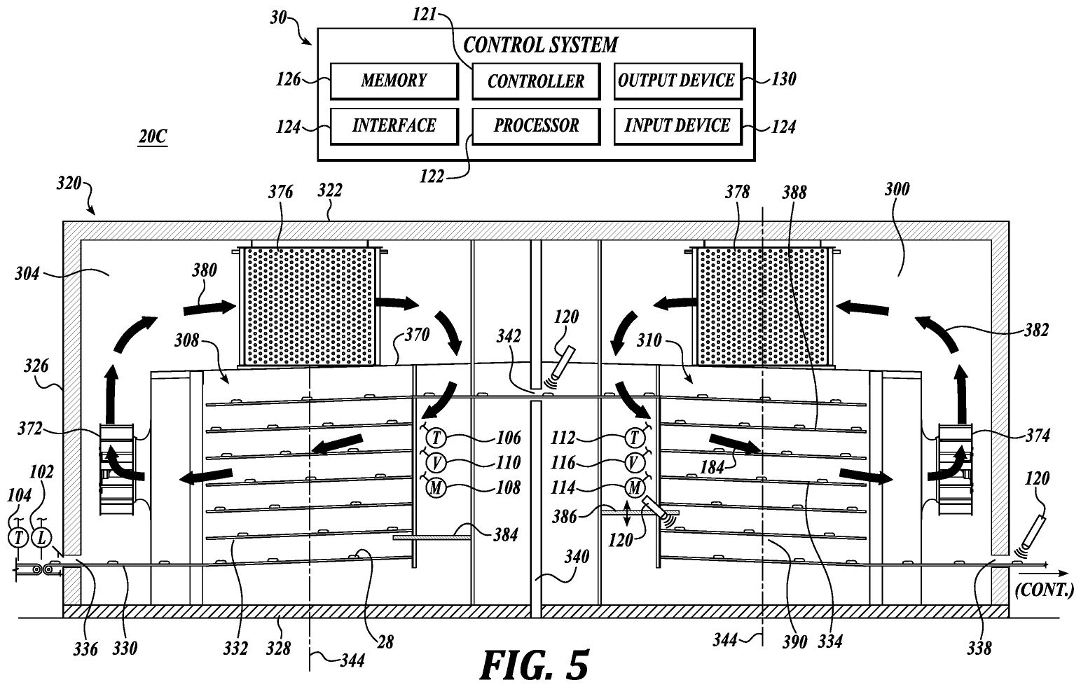

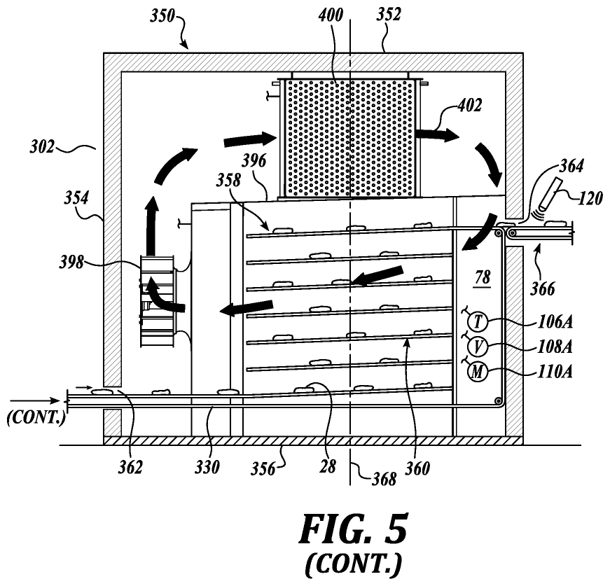

In existing food product production facilities, there may not be enough distance between the oven and freezer for the installation of a buffer chamber, such as chamber 140 shown in FIG. 2, or use a buffer conveyor or any significant length. A possible approach to this situation is shown in FIG. 5, wherein the thermal processing system 20C an existing freezer 300 functions as a buffer chamber and another freezer 302 is located downstream of the existing freezer. This could increase the capacity of an entire cook/freezer line, as well as improve yields, food quality, food safety, etc. Further, the existing refrigeration system of the original freezer/buffer chamber 300 could instead be operated as a heating system if it is determined that further heating of the food product is needed to achieve the desired level of lethality.

In addition, the original freezer/buffer chamber 300 can also be operated as a freezer to provide additional freezing capacity in case the additional capacity is needed due, for example, to an oven 304 having a higher capacity than the freezer 300. In this regard, optionally a mezzanine 386 similar to mezzanine 230 shown in FIG. 3, could be used to restrict the cooling to the section of the freezer below the mezzanine, so as not to interfere with the thermal equilibration occurring in the upper section of the freezer that has been converted to also function as a buffer chamber. In this situation, the flow of thermal fluid from the cooling unit 398 can be routed to beneath the mezzanine 386.

Describing the thermal proceeding system 20C in more specificity, FIG. 5 schematically shows the thermal processing system 20C, an oven 304 composed of an ascending spiral conveyor unit 308. The original freezer has been converted into a buffer chamber 300 that includes a descending spiral conveyor unit 310. A separate freezer 302 is located adjacent and downstream to the now buffer chamber 300. The freezer 302 includes an ascending spiral conveyor unit 360.

The oven 304 and the buffer chamber 300 are shown as being disposed in rectangularly shaped housing 320 having a top section or ceiling 322, longitudinal side sections or walls 324, and transverse end sections or walls 326, as well as a floor 328. The housing 320 is sized to contain the spiral or helical conveyor units 308 and 310.

An endless, continuous powered conveyor belt 330 for carrying food products through the system housing 320 is arranged in tiers forming an ascending spiral stack 332 in conveyor unit 308 and arranged in tiers forming a descending spiral stack 334 in conveyor unit 310. The conveyor belt 330 enters the spiral conveyor unit 310 at the bottom thereof by passing through an inlet opening 336 in wall 326, and then travels in a spiral path until reaching the top of the spiral stack 332, and then extends tangentially from the top of stack 332 to the top stack 334 to descend along the spiral conveyor unit 310, to eventually exit the unit buffer chamber 300 from the bottom tier of the stack 334 through outlet opening 338 formed in wall 326. The inlet 336 and outlet 338 openings can be substantially sealed from the ambient by air knives or other means.

A center or mid wall 340 divides the two spiral conveyor units 308 and 310 into separate oven and buffer compartments, wherein different process media conditions can be employed. For example, the temperature of the air, air/vapor mixture, steam, or other processing medium, the moisture content in the air, the medium velocity, etc., may be different in the two compartments created by the mid or center or cross wall 340. A close fitting opening 342 is provided in wall 340 to allow passage of the conveyor belt 330 and the food products being carried thereon. If needed, an air knife or similar/other sealing system can be used to provide a seal between the oven and buffer compartments.

The center of the conveyor stacks 322 and 334 extend around a central drive system that rotates the conveyor units 308 and 310 about a central axis 344. The drive system includes a cylindrical drive drum that frictionally and rotationally drives conveyor belt 330 over supports that are fixed in place exterior to the drum, thereby to rotate the belt about axis 344. The belt 330 tightens around the drive drum, creating enough friction therebetween to drive the belt forward to slide over the supports.

Describing the down steam freezer 302 in more detail, the freezer includes a generally rectangularly shaped housing 350 each having a top section or ceiling 352, longitudinal side sections or walls and end walls 354, as well as a floor 356. The housing 350 is sized to contain a spiral or helical conveyor 358.

The powered conveyor belt 330 also carries the work products 28 through the freezer 302 on a spiral conveyor unit 358 arranged in tiers forming an ascending spiral stack 360. The conveyor belt 330 enters the spiral conveyor unit 358 at the bottom thereof at an inlet 362 in wall 354 and then travels in a spiral path until reaching the top of the spiral stack 360, and then extends tangentially along the top of stack 360 to an exit opening 364 to deliver the food products to a takeaway conveyor 366. The section of the conveyor belt 330 located between the buffer chamber 300 and the freezer can be enclosed, for example, in a tunnel.

The center of the conveyor stack 360 extends around a central drive system that rotates the spiral conveyor unit 358 about a central axis 368. The drive system includes a cylindrical drive drum that frictionally and rotationally drives conveyor belt 330 over supports that are fixed in place exterior to the drum, thereby to rotate the belt about axis 368. The belt 330 tightens around the drive drum, creating enough friction therebetween to drive the belt forward to slide over the supports in a standard manner.

Referring back to the oven 304 and buffer unit 300, a top panel structure 370 overlies the conveyor stacks 308 and 310. Circulation fans 372 and 374 can be provided to be positioned at outward sides of the conveyor units 308 and 310 to draw processing medium, for example, air, across the interior of the conveyor stacks 332 and 334 (and around the drums) so as to thermally treat the food products being carried on the conveyor belt 330 and then direct such processing medium upwardly along the end walls 326 of the housing 320 toward the ceiling 322 of the housing. Thereafter, the processing medium is directed through heat exchangers 376 and 378 positioned on or above the top panel structure 370 of the oven 304 and buffer unit 300. The processing medium extends transversely across the top of each of the stacks 332 and 334. The heat exchangers 376 and 378 may be mounted on or just above the top structure 370 by an appropriate mounting structure.

The thermal processing air or other thermal processing medium being circulated by the fans 212 and 214, when passing through the heat exchanger 376 for the oven 304 is heated to a level as desired. For the buffer unit 300, the heat exchanger 398 may heat or cool the thermal processing air. The heated medium for the oven 304 and the heated medium for the buffer unit 300 flows horizontally over the top structure 210 until reaching the cross wall 342, wherein the processing medium is deflected downwardly to flow along the exposed adjacent portion of the conveyor stacks 332 and 334 and enter into the stacks in a lateral direction, as depicted by arrows 380 and 382, thereby thermal treating the food product primarily by convection heat transfer.

If the buffer unit is used to provide additional cooling, the cooling medium from the heat exchanger 378 is to be routed to below the mezzanine 386, so the flow pattern is not as shown by arrows 382, but rather the flow pattern will route the cooling medium to enter the stacks in a lateral direction at an elevation below the mezzanine 386.

The spatial arrangement of the fans 372 and 374, the heat exchangers 376 and 378, and optimally positioned wall 342 in relation to the respective stacks enables flow distribution to be uniform to each tier within the individual stacks to deliver an air flow mixture to approach the surfaces of the food items on the conveyor diagonally. This serves as a unique feature with respect to conventional twin-drum ovens manufactured by others.

Nonetheless, the fans 372 and 374 and the heat exchangers 376 and 378 can be arranged in other locations than as shown while satisfactorily distributing thermal processing fluid to the tiers of the stacks.

A first mezzanine 384 may be located in the lower portion of the oven chamber to effectively divide the conveyor stack 332 into a lower first processing zone below the mezzanine and an upper second processing zone above the mezzanine. The mezzanine extends outwardly from the mid wall 340 toward the conveyor stack 332. The central portion of the mezzanine is scalloped or otherwise relieved to provide clearance for the conveyor stack 332. The remainder of the mezzanine 384 (located outwardly of the conveyor stack 332) extends towards the center of the conveyor stack to approximately the location of the drive drum.

The first mezzanine 384 is shown as substantially planar in construction. Appropriate braces or reinforcements can be used to provide the needed structural integrity of the mezzanine.

Optionally, the mezzanine 384 is adjustable in elevation by an appropriate actuating system. Preferably, the actuation system is powered so the control system is able to alter the height of the mezzanine as desired. The first mezzanine 384 is shown as located at an elevation between the first and second lowest tiers of the conveyor stack 332. However, the mezzanine 384 can be positioned at a higher elevation, for example, at the level of the second lowest tier or at a level above the second lowest tier of the conveyor stack 332.

A second mezzanine 386 may be located in the lower portion of buffer chamber or compartment to effectively divide the conveyor stack 334 into an upper processing zone 388 if further processing (e.g., heating) of the product is needed and a lower buffer section or zone 390. If further processing of the food product is not required in chamber 300, then the entire chamber can serve as a buffer section or zone.

Or as discussed above, if additional cooling is desired, the lower zone 390 can be used to receive cooling medium from the heat exchanger 378.

The mezzanine 386 is shown as extending outwardly from the mid wall 340 toward the conveyor stack 334. The central portion of the mezzanine 386 is scalloped or shaped to provide clearance for the conveyor stack 334. The remainder of the mezzanine extends towards the center of the conveyor stack to approximately the location of the drive drum. In this manner, the mezzanine 386 prevents the processing medium, as depicted by arrows 382, from entering the ties of the stack 334 located below the mezzanine.

The second mezzanine 386 is also shown as substantially planar in construction. Appropriate braces or reinforcements can be used to provide the needed structural integrity of the mezzanine.

The mezzanine 386 may be adjustable in elevation, via an appropriate actuation system. Preferably, the actuation system is powered so the control system 30 is able to alter the height of the second mezzanine as desired. The second mezzanine 386 is shown as located at an elevation between the second and third lowest tiers of the conveyor stack 334. However, the mezzanine 386 can be positioned at a higher elevation, for example, at the level of the third lowest tier or above the third lowest tier. In this manner, the size of the buffer zone 390 can be adjusted so the length of time provided for the food items to equilibrate in temperature can be selected to achieve a desired kill level of the pathogens within the food products.

Next referring back to the freezer 302, a top panel structure 396 overlies the conveyor stack 360. A circulation fan 398 can be provided to be positioned at outward side of the spiral conveyor units 358 to draw processing medium, for example, air, across the interior of the conveyor stack 360 (and around the drum) so as to thermally treat the food products being carried on the conveyor belt 330 and then direct such processing medium upwardly along the end walls 354 of the housing 350 toward the ceiling 352 of the housing. Thereafter, the processing medium is directed through heat exchangers 400 positioned on or above the top panel structure 396. The processing medium extends transversely across the top of each of the stack 360. The heat exchanger 400 may be mounted on or just above the top structure 396 by an appropriate mounting structure.

The thermal processing air or other thermal processing medium being circulated by the fan 398, when passing through the heat exchanger 400 is cooled to a level as desired. The cooled medium for the freezer 302 flows horizontally over the top structure 396 until reaching the cross wall 354, wherein the processing medium is deflected downwardly to flow along the exposed adjacent portion of the conveyor stack 360 and enter into the stack in a lateral direction, as depicted by arrows 402, thereby thermal treating the food product primarily by convection heat transfer.

The spatial arrangement of the fan 398, the heat exchanger 400, and optimally positioned wall 354 in relation to the stack 360 enables flow distribution to be relatively uniform to each tier within the stack to deliver an air flow mixture to approach the surfaces of the food items on the conveyor diagonally. This serves as a unique feature with respect to conventional drum driven spiral conveyor ovens manufactured by others.

Although not shown, a mezzanine can be utilized with the freezer 304. The mezzanine can be similar in construction and operation to mezzanine 230 or 386 described above, but located toward the top of the stack 360. The mezzanine can provide the benefits of mezzanines 230 and 386. If a mezzanine is used with freezer 302, then the flow 402 of thermal processing fluid can be rerouted to be confined below the mezzanine, since the stack 360 is upwardly ascending.

As noted above, the buffer unit 300 can be heated or cooled as needed so as to reach the desired level of cooking of the food product, the desired level of the killing of pathogenic microorganisms on and/or within food products, and the desired level of freezing. As such, the heat exchanger 398 can be part of a refrigeration unit or heating unit or a heat pump and thus function as a source of thermal processing fluid for the buffer unit. Further, the heat generated when the buffer unit is being cooled can be routed to the oven 304, and correspondingly, the cooled medium generated when the buffer unit is being heated, can be routed to the freezer 302, thereby enhancing the efficiency of the system 20C.

As an alternative, the heat removed from the refrigerant during the operation of the freezer 302 can be used to supply heat to the buffer unit rather than seeking to dispel the heat via an evaporator. Further the cooled medium generated by the operation of the oven 304 can be used to cool the buffer unit 300 if the desired level of pasteurization has been reached, so that precooling of the food product can begin in the buffer unit.

As a further alternative, the buffer unit 300 can be configured so that heat is applied to the upper zone 388, and the resulting cooled medium can be used to cool the food product in the lower zone 390. In this regard, a heat pump could be used to achieve this result.

To illustrate a potential advantage of this alternative, in a current system for cooking food products and the quickly freezing the cooked product, the thermal medium in the oven may be at 375° F. and −40° F. in the freezer. In the thermal processing system 20C that does not utilize a heat pump, the food thermal processing medium may be 375° F. in the oven, 80° F. in the buffer unit 300 and −40° F. in the freezer 302. If a heat pump is used in conjunction with the thermal processing system 20C, it is possible that the processing medium is 375° F. in the oven, 170° F. in the upper zone 388 of the buffer unit, 40° F. in the lower zone 390 of the buffer unit and −40° F. in the freezer. In this case, the time spent in the 350° F. oven and the −40° F. freezer can be significantly shortened, thereby increasing the efficiency of the thermal processing of the food items. Also, the quantity of food product that can be processed per unit time by the oven and freezer can be significantly increased.

As can be appreciated, the thermal processing system 20C can be achieved at a far lesser cost than installing a new system that has higher capacity and/or matches better the capacities of the oven and freezer. As discussed above, by the expediency of adding a new freezer and operating the original freezer as an oven or a freezer as needed, as well as operating as a buffer, the capacity of the overall system 20C can be significantly increased.

As in the thermal processing systems 20, 20A, and 20B, one or more thermal imaging cameras 120 may employed in thermal processing system 20C to serve the same function as in processing systems 20, 20A, and 20B, including to determine the external and internal temperature of the food products being processed. The thermal cameras 120 can be located, for example, in at the entry to the buffer chamber, in the buffer chamber at a location downstream of the second mezzanine 386, at the exit from the buffer chamber 330, at the exit from freezer 302. Of course, not all of these thermal imaging cameras may be used, and/or the thermal imaging cameras may be positioned at other locations.

As in the thermal processing systems 20, 20A, 20B, a control system 30 controls the operation of the thermal processing system 20C in the manner described above, including, for example, the speed of the conveyor 330 and the operation of the heating and cooling systems and heat pump if utilized. Additionally, the control system controls the level of the mezzanines, and thus the time the food products are located in the lower zone 390. As in the thermal processing systems 20, 20A, 20B, the control system 30 can be hard wired to the components of the thermal processing system 20C or may be wirelessly interconnected to the components.

As discussed above, for example, relative to thermal processing system 20, inputs to the control system 30 of the thermal processing system 20C can be from a measuring system that employs the same or similar load, temperature, thermal medium velocity and moisture measuring instruments or devices as discuss able. Such instruments and devices are identified with the same part numbers as used above, or with the addition of the suffix “A.” As such, the above description will not be repeated here.

As will be appreciated, in addition to serving as a buffer chamber 300, the chamber can be used to increase the cooking capacity of the system 20C or increase the cooling capacity of the system 20C. Not uncommonly, the cooking/heating capacity of a thermal processing system is not evenly matched with the freezing/cooling capacity of the system. The required capacity for heating/cooking can depend on, for example, the type of food product being processed, size of the food product being processed, the variation in the size of the food product being processed, the type of coating, if any, applied to the food product, the throughput of the food product desired, the initial temperature of the food product before entering the oven, the desired final desired temperature of the food product, the desired time/temperature profile of the food product, the desired safety margin in the pathogen lethality achieved, These factors may also affect the required capacity for the needed cooling/freezing of the food product. Also, the inherent capacity of heating/cooking oven may not match the inherent capacity of the cooler/freezer. This disparity can be alleviated or even eliminated by the buffer chamber when operated to add heat or cooling to the food product in the chamber.

Various coatings may be applied to the food product that affect the evaporative cooling on the surface of the food product. Such coating can be of many different types, and more than one type of coating can be used. A few types of coatings include flours; batters, beaten egg, starches, bread crumbs, panko,

As noted above, the thermal processing systems 20, 20A, 20B, and 20C of the present disclosure seeks to take into consideration the elevated temperature within the interior of the food product after leaving the oven, and thus the continued killing of pathogenic microorganisms within food products. Thus, the thermal processing of the food product continues beyond the point in time that the food product leaves the oven to include the time that the food product is thermally equilibrating. This leads to several advantages, including increased product throughput since the food product is removed from the oven at an earlier stage than has conventionally been the practice. Also, overcooking of food product is reduced, so more moisture is retained in the food product, thereby improving the quality of the food product as well as its net weight. In addition, less energy is expended in cooking the food product. These advantages are achieved while at the same time the food product is safely pasteurized to achieve a desired kill level of the pathogenic microorganisms within the food product.

Further, heated food products, upon exiting an oven, often produce large amounts of steam that ends up as frost on the coils of the freezers and on the conveyor belts. The frost can cause downtime, thereby interrupting the product schedule due to the need to defrost the coils and belt so that the freezer retains enough capacity to effectively freeze the food products. A lower cooking temperature of the food products will reduce the amount of steam generated and reduce the frequency of needing to defrost the freezer and/or conveyor.

In addition, if product is in the equilibrium chamber for some period of time, the steam can be removed from the product and chamber prior to entering the food product into the freezer. Overall, the loss of efficiency and downtime for freezers due to frost can be greatly reduced by the systems and methods of the present disclosure.

Also, since less thermal/heat energy is used to heat/cook the food product, less heat energy needs to be removed from the food product in the freezer. As such, the time required to cool the food product to a desired temperature is reduced. This adds to the throughput of the thermal processing system, as well as reduces the cost of operating the freezer.

Further, utilizing the post oven equilibration time period to continue to eradicate pathogenic microorganisms is applicable to not only the situation wherein individual food products are positioned apart from each other when being conveyed through the processing systems 20, 20A, 20B, and 20C, but also in situations where the food product is loaded as overlapping or even as a continuous layer of food product, for example vegetables or smaller meat items, such as chicken wings. In this situation the heat from one food product may be advantageously transferred to adjacent food products to enable the entire food mass to reach the same equilibration temperature. This would help avoid overcooking smaller/thinner food items and undercooking larger food items.

While illustrative embodiments have been illustrated and described, it will be appreciated that various changes can be made therein without departing from the spirit and scope of the invention.

For example, a thermal imaging camera 120 can also be positioned at the end of the buffer conveyor 26, the buffer chamber 140, and the end of the buffer 160 to determine the external and internal temperature of the food product at this point in time of the thermal processing of the food product. This thermal imaging camera 120 can be in lieu of, or in addition to, the thermal imaging camera 104. In this regard, it may not be desirable to place a thermal imaging camera in the oven 22A due to the harsh environmental conditions in the oven, so the temperature determination within the interior of the food product from thermal imaging camera 120 is relied upon.

The thermal image of the food product from the second thermal imaging camera 120 is also provided to an image segmentation machine learning model that segments the thermal image to identify pixels that depict the food product. Features extracted from the segmented thermal image (including, but not limited to, the measured surface temperature of the food product) are provided to a temperature prediction machine learning model in order to predict the internal temperature of the food product. Training data for the temperature prediction machine learning model may be obtained by using temperature probes to generate ground truth internal temperature information. The temperature probes may be placed into the food products after exiting oven 22 or before entering the ovens 22 and 22A.

Regardless of the placement of the thermal imaging cameras, the camera can be used to determine the temperature of all of the food product being processed or can be used to statistically sample the food product being processed. In this latter case, for example, the cameras can periodically select the food product to be measured based on one or more attributes, such as the size of the food product, the thickness of the food product, the volume of the food product, and measure its temperature and use that information to predict the internal temperature of the food product as well as predict the change in the internal temperature the food product over time. This information can be used to determine the log kill of pathogens on and/or in the food product.

Moreover, the temperature probes may monitor the temperature change over time within the interior of the food product to ascertain how the temperature of the food product equilibrates over time, upstream from the second thermal imaging camera 120. This information can be associated with the thermal image captured by the second thermal imaging camera. In this manner, the predicted temperature equilibration of the food product based on the information from the first thermal temperature camera can be confirmed. This information can be used to analyze and confirm on a time temperature basis the pathogenic organism kill level achieved during the thermal processing of the food product prior to the second thermal imaging camera. Further, this information can be used by the control system 30 to adjust one or more of the various operating parameters of thermal processing systems 20, 20A, 20B and 20C.

As another example, the thermal imaging cameras discussed above may be replaced by a probe placed into to food product just after exiting the oven 22. The probe may be placed into the food product manually or via an automatic actuator system, as disclosed in U.S. Pat. No. 9,366,580 B2, which is incorporated by reference herein. The temperature data from the probe may be transmitted wirelessly to an external receiver connected to the control system 30. In this manner, the expected continued increase in temperature of the interior of the food product can be monitored and also the kill level of the pathogens within the food product can be determined. This information can be used to set as well as adjust the operating parameters of the oven and/or buffer.

Further, the probe may be retained in the food product during further processing, such as freezing. In this manner, the information from the probes may be used to set and/or adjust the operating parameters of the freezer.

In addition, the probe may include a series of thermocouples along its depth, so as to measure the temperature of the food products at various depths from the exterior of the food product to central interior of the food product. This enables the speed and extent of thermal equilibration of the food product to be monitored.

As will be appreciated, the use of temperature probes to measure and monitor the temperature of the food item after leaving the oven would be used on a sampling basis, whereas with the use of a thermal imaging camera all of the food products being processed could be monitored. Nonetheless, the use of the temperature probes can provide a substantially accurate analysis of the food products being processed, including if the desired kill level of the pathogenic organisms within the food products is being achieved.

Also, as noted above, the temperature probe may be placed into the food product even before entering the oven. As a result, the temperature rise within the interior of the food product can be monitored both while within the oven as well as after exiting the oven while thermal processing of the food item continues to occur during the temperature equilibration phase. As a result, an accurate time temperature analysis of the food product being monitored can be determined. Further, as also noted above, the temperature probe may be retained in the food product after temperature equilibration, for example, while being cooled and frozen. This can help determine when the desire freezing temperature has been reached, which information can be used to help control the operation of the freezer.

As a further example, the ovens 22, 22A, and 304 described above are spiral ovens, and freezers 24, 300, and 302 described above are spiral freezers. Spiral ovens and freezers have the advantage that the length of the spiral conveyor within the oven or freezer structure can be relatively long relative to the footprint of the oven/freezer. As a result, large quantities of food product can be processed while requiring a relatively small floor area. However, the present system and process can be carried out using other types of ovens and/or freezers, including convection ovens, impingement ovens/freezers, microwave ovens, radio frequency ovens, whether or not the ovens/freezers utilize a built-in conveyor.

### Example 1

Two chicken fillets of substantially the same size and weight were placed in an oven after a first temperature probe was placed in the thickest location of the fillet and a second temperature probe was placed at a thin tail location, thereby to constantly measure the temperatures at these locations on fillet. The oven temperature was set to 375° F. The fillet labeled as “A” was removed from the oven when the first probe reached 150° F., which was 22 minutes after being placed into the oven. The temperature of the second probe at the tail location was at about 204° F. The fillet labeled as “B” was removed from the oven when the first probe reached 170° F., which occurred at 31 minutes. This is 9 minutes later than fillet A, so for fillet A about 40% of the heating requirement was saved.

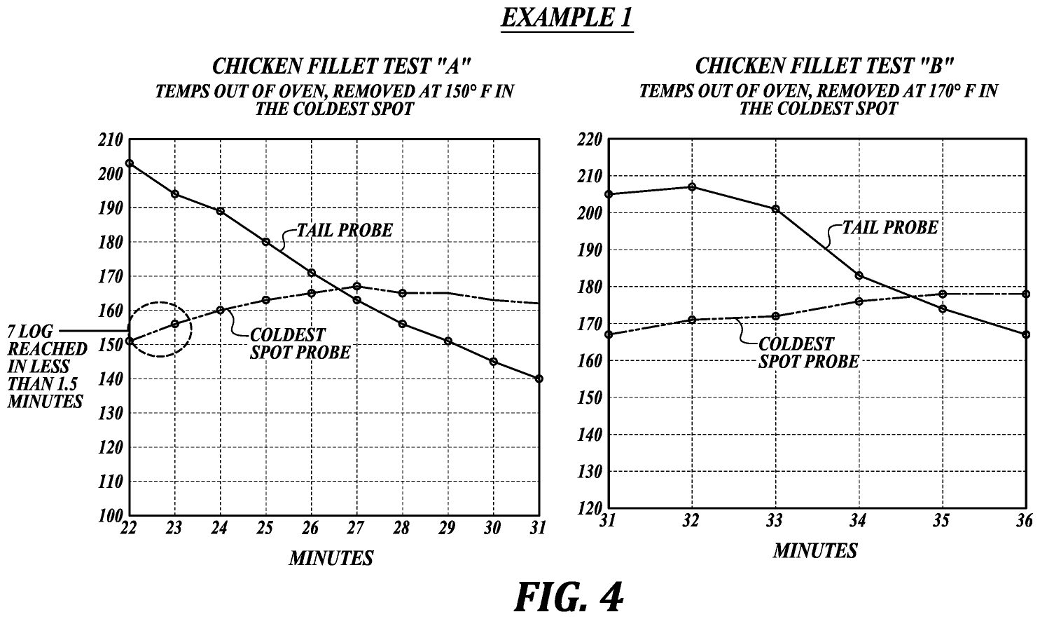

As shown in FIG. 4, the internal temperature of fillet “A” continued to increase and reached a 7 Log kill level within 1.5 minutes after removal from the oven. Further, the internal temperature reached 165° F. after 4 minutes. As noted above, at 165° F. microorganisms are instantly killed. As would be expected, the thin tail section of the fillet, having already reached maximum temperature, reduced in temperature after leaving the oven.

As also shown in FIG. 4, the internal temperature of fillet “B” also continued to rise after removal from the oven, though at a slower rate than the temperature rise of fillet “A” after removal from the oven. The temperature of the tail section of fillet “B” also reduced after leaving the oven.

There was little difference between fillet “A” and “B” in appearance, texture, or taste.

This example demonstrated that fillet “A” achieved an acceptable lethality of pathogenic microorganisms even though removed from the oven 10 minutes earlier than fillet “B.” This resulted in a significant savings of not only cooking time in the oven, but also energy expended.

The embodiments of the invention in which an exclusive property or privilege is claimed are defined as follows:

1. A system for thermal processing of food products:
an oven for heating food products, the oven operating under selected operating parameters;
a buffer area operating under selected operational parameters for receiving and holding food products heated in the oven for a selected time period before further processing of the food products;
a temperature determining system comprising at least one temperature measuring device for measuring the temperature of the food product during the thermal processing of the food product and producing data related thereto;
a control system comprising a computing device, the control system:
operational to receive the operational parameters of at least one of the oven and the buffer area, and to receive the temperature measurement data;
operational to use the temperature measurement data to determine over time the temperature equilibration of the interior of the food product after leaving the oven to model the lethality of pathogenic microorganisms in the interior of the food product; and
operational to adjust one or more of the operational parameters of at least one of the oven and buffer areas as needed to achieve a desired lethality of the pathogenic microorganisms in the interior of the food product.

2. The system of claim 1, wherein the temperature determining system determines the temperature of the food products at one or more of the following locations:
before entering the oven;
within the oven;
after exiting the oven;
within the buffer area;
at the buffer area exit;
exterior to the buffer area.

3. The system of claim 1, wherein the temperature determining system comprises at least one thermal imaging camera.

4. The system of claim 3, wherein the control system processes the data from the at least one thermal imaging camera and uses past temperature measurements of food products that had been analyzed using a machine learning model to determine the internal temperature of the food product and the equilibration of the internal temperature of the food product over time.

5. The system of claim 2, wherein the thermal determining system comprises a thermal imaging camera capturing the food products in the buffer area.

6. The system of claim 5, wherein the control system processes the data from the thermal imaging camera and also uses past temperature measurements of food products that had been analyzed using a machine learning model to determine the internal temperature of the food product and the equilibration of the internal temperature of the food product occurring prior to capture by the thermal imaging camera.

7. The system of claim 2, wherein the temperature determining system comprises at least one probe for determining the internal temperature of the food product.

8. The system of claim 1, further comprises a conveyance to carry the food product through the oven and through the buffer area.

9. The system of claim 8, wherein the control of the speed of conveyance of the food product through the buffer area is independent of the control of the speed of conveyance of the food product through the oven.

10. The system of claim 1, wherein the operational parameters of the oven comprise the temperature within the oven, the humidity within the oven, the composition of the processing medium in the oven, the temperature of the processing medium entering the oven, the volumetric flow rate of the processing medium flowing through the oven, the duration of time of the food product within the oven, the pressure within the oven, the dew point temperature within the oven, and the oxygen content within the oven.

11. The system of claim 1, wherein:
the buffer area comprises a holding chamber; and
the conveyance carries the food product through the holding chamber.

12. The system of claim 11, further comprising a source of thermal processing fluid for supplying the holding chamber.

13. The system of claim 12, wherein the temperature of the thermal processing fluid for the holding chamber is above or below 32 degrees F.

14. The system of claim 12, wherein the source of thermal processing fluid comprises a heating system and cooling system for supplying heated and/or cooled thermal processing fluid to the holding chamber.

15. The system of claim 14, wherein the holding chamber divided into a heating zone and a cooling zone, wherein the heating system and cooling system supplies heated thermal processing fluid to the heating zone and/or supplies cooled thermal processing fluid to the cooling zone.

16. The system of claim 1, further comprising a freezer located downstream of the buffer area, the freezer operating under selected operational parameters.

17. The system of claim 16, wherein the control system controlling the operational parameters of the freezer.

18. The system of claim 16, wherein heat generated during the operation of the freezer is supplied to the buffer area.

19. A time-temperature method for thermal processing of a food product:
subjecting the food product to thermal processing in an oven operated under selected operating parameters;
transferring the food products to a buffer area operated under one or more selected operating parameters;
removing the food product from the buffer area for further processing;
determining the temperature of the food product during the thermal processing of the food product, including the internal temperature of the food product, over time after being heated in the oven;
modeling a lethality of pathogenic microorganisms on and/or in the food products after being heated in the oven to determine if a desired level of lethality of pathogenic microorganisms was achieved, the modeling using the determined internal temperature of the food product after leaving the oven;
based on such modeling, if the desired level of lethality of pathogenic microorganisms on and/or in the food products was not achieved, adjusting one or more operating parameters of the oven and/or the buffer area.

20. The method of claim 19, further comprising supplying a thermal processing medium to the buffer area to assist in achieving a desired level of lethality of the pathogenic microorganisms.
